(19) United States
(12) Patent Application Publication
(10) Pub. No.: US 2023/0386636 A1
(43) Pub. Date: Nov. 30, 2023

(54) Treatment Support

(71) Applicant: Closed Loop Medicine Ltd., Cambridge (GB)

(72) Inventors: Paul Goldsmith, London (GB); Adrian Williams, Cambridge (GB); David O'Regan, London (GB); David Cox, London (GB)

(73) Assignee: Closed Loop Medicine Ltd., Cambridge (GB)

(21) Appl. No.: 18/311,487

(22) Filed: May 3, 2023

Related U.S. Application Data
(63) Continuation of application No. PCT/GB2021/052858, filed on Nov. 4, 2021.
(60) Provisional application No. 63/109,668, filed on Nov. 4, 2020.

(51) Int. Cl. G16H 20/10; G16H 10/60

(52) U.S. Cl. CPC G16H 20/10; G16H 10/60

(57) ABSTRACT

A computer implemented method for supporting the treatment of insomnia with melatonin. The method includes receiving sleep data relating to a patient; determining a dosage time for the patient to take a dose of the melatonin based on the sleep data; and indicating the dosage time.

### BACKGROUND

Insomnia is a dissatisfaction with sleep quantity or quality. One third of the population at some point in the year have some difficulties in getting to sleep, staying asleep, or waking early. Insomnia is typically followed by daytime sleepiness, low energy, irritability, and a depressed mood. The physiology of that one third of the population is measurably different than the other two thirds with adrenaline and cortisol levels statistically elevated making a “hyper-arousable” physiology. In one third of that one third (therefore one in ten people) the problem carries on because it can become a learned pattern of behavior for which treatment revolves around unlearning that behavior.

There are many factors and causes linked to insomnia: sleep breathing disorders, poor sleep hygiene, restless legs syndrome, hormone shifts, life events such as fear, stress, anxiety, emotional or mental tension, work problems, financial stress, birth of a child, and bereavement, disturbances of the circadian rhythm, such as shift work and jet lag, increased exposure to the blue light from artificial sources, such as phones or computers, gastrointestinal issues such as heartburn or constipation and many more.

Various treatment options are currently available, dependent on the type of insomnia. Typically though for the commonest psychophysiological insomnia, the treatment options are around behavioral modification, with or without pharmacotherapy. Behavioral modification involves improving one's “sleep hygiene”, such as limiting caffeine, limiting late night exposure to light, exercising at appropriate times and getting up at a regular time.

Melatonin is a naturally occurring hormone released by the pineal gland that regulates the sleep-wake cycle. Previous research suggests that supplementation with melatonin may help increase total sleep time in individuals suffering from insufficient sleep or altered sleep schedules, although evidence to support this is not strong. This may be, at least in part, due to the many different types of influencing factors and causes of insomnia that mean that “one size fits all” generic treatments currently available are unlikely to be effective across a range of different subjects suffering from insomnia.

### SUMMARY

The present invention addresses the described issues with current insomnia treatments by providing a personalized treatment approach that takes into account the various influencing factors affecting a subject suffering from insomnia. This results in a more effective therapy than those existing currently.

According to a first aspect of the present disclosure there is provided a computer implemented method for supporting the treatment of insomnia with melatonin, the method comprising:
receiving sleep data relating to a patient;
determining a dosage time for the patient to take the melatonin based on the sleep data; and
indicating the dosage time.

According to a second aspect, a system for supporting the treatment of insomnia with melatonin is provided, the system comprising a processor configured to perform the method of any preceding claim.

According to a third aspect, there is provided a computer program product storing computer executable instructions for performing the method as defined in the first aspect.

According to a fourth aspect, melatonin is useful in the treatment of insomnia, wherein the melatonin dosage regimen is determined by steps comprising:
receiving sleep data relating to a patient;
determining a dosage time for the patient to take the melatonin based on the sleep data; and
indicating the dosage time.

According to a fifth aspect, there is provided a method of treating insomnia comprising administering melatonin to a patient in need thereof, wherein the melatonin dosage regimen is determined by steps comprising:
receiving sleep data relating to a patient;
determining a dosage time for the patient to take the melatonin based on the sleep data; and
indicating the dosage time.

According to a sixth aspect, melatonin is used in the manufacture a medicament for the treatment of insomnia, wherein the melatonin dosage regimen is determined by steps comprising:
receiving sleep data relating to a patient;
determining a dosage time for the patient to take the melatonin based on the sleep data; and
indicating the dosage time.

According to a seventh aspect, a kit comprises melatonin and instructions for use, wherein the instructions for use comprise indicating the use of a computer implemented method according to any one of claims 1 to 35.

While the disclosure is amenable to various modifications and alternative forms, specifics thereof have been shown by way of example in the drawings and will be described in detail. It should be understood, however, that other embodiments, beyond the particular embodiments described, are possible as well. All modifications, equivalents, and alternative embodiments falling within the spirit and scope of the appended claims are covered as well.

The above discussion is not intended to represent every example embodiment or every implementation within the scope of the current or future Claim sets. The figures and Detailed Description that follow also exemplify various example embodiments. Various example embodiments may be more completely understood in consideration of the following Detailed Description in connection with the accompanying Drawings.

### BRIEF DESCRIPTION OF THE DRAWINGS

One or more embodiments will now be described by way of example only with reference to the accompanying drawings in which:

FIG. 1 illustrates a computer implemented method according to an embodiment of the present invention;

FIG. 2 illustrates a system for performing any of the computer implemented methods disclosed herein; and

FIG. 3 illustrates a user interface sequence suitable for display on the patient device when the system is carrying out any of the computer implemented methods disclosed herein.

### DETAILED DESCRIPTION

The first aspect of the disclosure provides a computer implemented method for supporting the treatment of insomnia with melatonin. The method comprises receiving sleep data relating to a patient, determining a dosage time for the patient to take a dose of the melatonin based on the sleep data, and indicating the dosage time.

The term “treatment” includes the amelioration of the disease or condition, or a symptom or symptoms thereof. Treatment also includes the amelioration of the side-effects of another therapy, such as a pharmacological therapy. Treatment also includes the reduction in a patient's dependence on another pharmacological drug, or behaviour. “Amelioration” is an improvement, or perceived improvement, in the patient's condition, or a change in a patient's condition that makes it, or side-effects, increasingly tolerable.

The computer implemented method may be implemented on a system comprising one or more processing devices. Example systems may include a single device such as a computer or a mobile device such as a patient mobile device. Example systems may also include a plurality of physically distributed devices connected over a network such as a cellular network or the internet. For example, a plurality of distributed devices could include a patient device, a server and optionally a clinician device.

The term “supporting” relates to the supplementary aid of an existing treatment of insomnia with melatonin. For example, the method may inform a clinician on how to optimise the treatment of insomnia with melatonin by indicating a recommended dosage time. In other examples, the method may indicate an optimum dosage time to a clinician to specifically direct the clinician to optimise the treatment by prescribing the dosage time to the patient. In other examples, where a patient has an existing melatonin prescription, the method may support treatment by directly indicating a dosage time of the melatonin to the patient.

The treatment of insomnia may relate to different types of insomnia. For example, the insomnia may relate to any of: delayed sleep phase insomnia (referred to herein as group A); early morning wakening insomnia (referred to herein as group B); and sleep maintenance insomnia (referred to herein as group C). Patients may suffer from more than one type of insomnia. In a particular embodiment, if a patient with Group A insomnia plus B or C, then the treatment approach for Group A overrides. Group A insomnia (delayed sleep phase) may be defined as patients with a delayed sleep phase component to their insomnia of greater than a 1 hour delay, more notably a 2 hour delay, between a target sleep onset time and their actual sleep onset time. The target sleep onset time may relate to an optimal long term negotiated sleep onset time. Therefore, group A relates to patients who have a greater than 2-hour delay in sleep onset time from their target or optimal, “sensible” sleep onset time. Group A does not encompass patients who go to bed much earlier (e.g. 9 pm) than their optimal sleep onset time (e.g. 11 pm) in a desperate attempt to obtain prolonged sleep.

“Patient” and “subject” are used interchangeably and refer to the subject that is to receive the melatonin. Preferably the subject is a human.

The method includes receiving sleep data relating to a patient. The sleep data may comprise any data relating to a patient's sleep. The sleep data may comprise one or more of: wake-up time data, sleep duration, sleep onset time, sleep quality, number of waking episodes, duration of waking episodes and day-time sleep data.

The sleep data may comprise actual sleep data comprising recorded data of actual sleep parameters of the patient. The actual sleep data may comprise one or more of: an actual wake-up time, actual sleep duration, actual sleep onset time, actual sleep quality, actual number of waking episodes, actual duration of waking episodes and actual day-time sleep duration.

The sleep data may comprise target sleep data comprising target data that a patient should adhere to in order to progress to one or more patient goals (discussed further below). The target sleep data may comprise: a target wake-up time, also referred to as an anchored wake-up time, a target sleep duration, a target sleep hunger and a target sleep onset time.

The sleep data may comprise patient goal data comprising desired sleep data of the patient. The patient goal data may comprise one or more of: a desired wake-up time; a desired sleep duration; a desired sleep onset time; a desired sleep quality; a desired number of waking episodes; a desired duration of waking episodes and a desired day-time sleep duration. In other examples, the patient goal data may be received as further patient data.

It will be appreciated that the sleep data may comprise one or more other sleep related parameters. For example, the sleep data may comprise one or more parameters derivable from the above listed parameters such as sleep efficiency, equal to the actual sleep duration divided by the actual time spent in bed.

Receiving the sleep data may comprise receiving the sleep data by patient or clinician input. For example, a patient may input or record actual sleep data following waking up in the morning. A patient may input their patient goal data at the beginning of treatment support or may update the patient goal data as the treatment support progresses. A clinician may input initial sleep data when commencing the treatment support. Systems implementing the computer implemented method may provide a user input module, such as a keyboard, touchscreen device or other apparatus as known in the art. For example, a patient may input actual patient data or patient goal data via a touchscreen of a mobile device.

Receiving sleep data may also comprise receiving sleep data from one or more sensors. The one or more sensors may comprise sensors capable of monitoring, recording and/or assessing a patient's sleep experience. The one or more sensors may include a motion sensor, a video camera, a microphone, an electroencephalogram (EEG), a heart rate monitor, a pulse oximeter, or any other sensor capable of gathering polysomnographic data. The one or more sensors may be worn by the patient to whom the sleep data relates.

Receiving the sleep data may comprise receiving the sleep data from one or more sensors forming part of a system implementing the method, for example an accelerometer on a smart watch. Receiving the sleep data may comprise receiving the sleep data from one or more sensors which may be external to the system, for example a separate medical device. The method may include communicating with the one or more sensors to receive the sleep data. The method may include processing raw sensor data to generate the sleep data.

The method may also comprise receiving sleep data by retrieval from a local or networked storage source. For example, the method may comprise receiving medical record data from a network storage source.

The method may also comprise storing the sleep data in a local or networked storage source. The method may further comprise storing any data required by or produced in the performance of the method. For example, the method may store further patient data, determined dosage times, actual dosage times, processing errors or any other relevant data.

The method further includes determining a dosage time for the patient to take a dose of the melatonin based on the sleep data.

The dosage time may relate to a time of day, for example 9 pm, or may be a time relative to a parameter of the sleep data, for example 16 hours after actual wake-up time. The dosage time may relate to a single daily dose or may comprise multiple dosage timings relating to different times throughout the day.

The step of determining the dosage time is discussed further below.

The method further comprises indicating the dosage time. In some examples, indicating the dosage time may comprise indicating the dosage time directly to a patient. Determining the dosage time and indicating the dosage time to a patient may comprise determining the dosage time and indicating the dosage time on a daily basis. Determining the dosage time on a daily basis may comprise determining the dosage time based on actual sleep data from a previous night's sleep or from several previous nights sleep (for example based on moving averages).

In some examples the method comprises indicating the dosage time to a clinician. Indicating the dosage time to the clinician may be for informing the clinician of a recommended dosage time or for instructing the clinician to prescribe the dosage time (and optionally a bedtime/target sleep onset time as discussed further below). Indicating the dosage time to the clinician may be done on an ad-hoc basis or a regular basis, such as weekly, bi-weekly or monthly etc. In such examples, determining the dosage time may be based on averaged actual sleep data for the intervening period. Alternatively, or in addition, determining the dosage time may comprise determining a dosage time on a daily basis and indicating an average daily dosage time to the clinician.

Therefore, in the discussion that follows all types of data, including sleep data, particularly actual sleep data, and any further patient data may comprise daily data such as daily actual sleep data, corresponding to data for a particular day (for example a specific actual wake-time on the particular day) or to average data, such as average actual sleep data, corresponding to a plurality of days (for example an average actual wake-time for the plurality of days). The method may comprise determining a moving average (such as a moving 3-day average or moving 7-day average) of daily data for any of the data types measured, received, determined or indicated by the method (such as sleep data, actual sleep data, dosage times or any further patient data). Although the data is described as daily, it should be understood that the precision of timings of specific data types can comprise timing resolution that is accurate to minutes or seconds or better.

Returning to the step of determining the dosage time, determining the dosage time may be based on one or more parameters of the sleep data. In one example, determining the dosage time may be based on wake-up time data. The wake-up time data may comprise the actual wake-up time and/or the target wake-up time. The target wake-up time may be referred to as an anchor wake-up time. The method may determine the dosage time based on the target wake-up time in a way that anchors this target wake-up time in an attempt to anchor the patient's sleep pattern over multiple days. In this way, the method can anchor or fix the target wake-up time regardless of a patient's sleep on a particular day in an attempt to synchronise the patient's long term sleep pattern with an actual wake-up time trending towards the target wake-up time.

To understand the wake time anchoring concept further we introduce two major drivers of sleep onset:
1. The sleep homeostat, also referred to as ‘sleep drive,’ ‘sleep hunger’ or ‘sleep debt’. This sleep hunger can be approximated as the length of time the patient has been awake.
2. The circadian drive. In particular, a rise in natural melatonin levels opens up a window of opportunity for sleep.

Sleep initiation or sleep onset is most likely when 1 and 2 are synchronised.

The remainder of the natural melatonin nocturnal secretion is believed to facilitate nocturnal occurrences, in humans this being normal (non-insomnia) sleep.

The method according to the first aspect comprises determining the dosage time of exogenous melatonin, or treatment with melatonin, such that the subsequent rise in melatonin levels (from a low baseline) align with a time of sufficient sleep hunger to initiate sleep. The required level of sleep hunger may be greater than ‘normal’ to overcome counteracting arousing factors of insomnia related anxiety that can be contributing to the insomnia in the first place. In other words, the patient may need a higher sleep hunger and therefore to go to bed later than they would in the absence of their insomnia or in a ‘cured’ state.

Therefore, the method can support the treatment of insomnia by controlling sleep hunger. The method can control sleep hunger by anchoring an awakening time. The method can anchor the awakening time by receiving a target wake-up time and an actual wake-up time and determining a dosage time based on the target wake-up time and the actual wake-up time. In one or more examples, the method may comprise determining a dosage time as a fixed time period following an actual wake-up time or the target wake-up time depending upon which time occurs first.

The method may comprise determining the dosage time based on the target sleep onset time. In some examples, the sleep data may comprise the target sleep onset time (for example as received from patient and/or clinician input). In some examples, the method may comprise determining the target sleep onset time based on the target wake-up time and/or the actual wake-up time. For example, the method may determine the target sleep-onset time to correspond to a fixed time period following an actual wake-up time or the target wake-up time depending upon which time occurs first. The fixed time period may correspond to a sufficient sleep hunger. The method may determine the dosage time as a further fixed time period before the target sleep-onset time.

To illustrate an example, the method may support the treatment of insomnia with melatonin for group A insomnia. The method may comprise receiving a target wake-up time and an actual wake-up time. The method may comprise determining the dosage time as from 8 to 12 hours, preferably 10 hours, after the actual wake-up time or the target wake-up time. In some examples, determining the dosage time comprises determining the dosage time as from 8 to 12 hours, preferably 10 hours, after the actual wake-up time or the target wake-up time, whichever is later. For a stable anchored wake-up time (actual wake-up time˜target wake-up time) 10 hours after the actual wake-up time corresponds to a dosage time for taking the melatonin that is 12 hours prior to the nadir of the patient's nocturnal temperature, which is 2 hours prior to actual wake time. Therefore, for a stable anchored wake-up time, the dosage time corresponds to 14 hours prior to target wake-up time.

To illustrate a further example, the method may support the treatment of insomnia with melatonin for group B or C type insomnia. The method may comprise receiving a target wake-up time and an actual wake-up time. The method may comprise determining the dosage time as from 11 to 19, preferably 15 hours after the actual wake-up time or the target wake-up time. In some examples, determining the dosage time comprises determining the dosage time as from 11 to 19 hours, preferably 15 hours, after the actual wake-up time or the target wake-up time, whichever is later. A dosage time of 15 hours after the target wake-up time corresponds to 24 hours minus a calculated average daily sleep time for that patient (8 hours in this example) minus 1 further hour relating to taking the melatonin 1 hour prior to sleep onset time (a dosage offset time discussed further below).

Therefore, more generally, the method may comprise determining the dosage time based on the actual wake-up time, the target wake-up time, a target sleep duration and a dosage offset time. For example, determining the dosage time may comprise determining the dosage time, t_{dose}, as the (later of) actual wake-up time, t_{AW}, or target wake-up, t_{TW}, time plus 24 hours minus the target sleep duration, t_{TSD}, minus the dosage offset time, t_{DO}:

t_{dose}=later of(t_{AW},t_{TW})+24−t_{TSD}−t_{DO}

The target sleep duration may be based on an average sleep duration for the patient, or a ‘normal’ sleep duration for the patient (for example 8 hours). However, the normal or average sleep duration follows a normal distribution for the population generally and a normal sleep duration can be personal to the patient. The method may determine the target sleep duration based on the sleep data or the further patient data. The target sleep duration may be based or equal to the desired sleep duration.

The dosage offset time defines a time between the dosage time and a target sleep onset time or target bedtime. The dosage offset time may be between 0 and 3 hours and may preferably be 1 hour. By taking melatonin 1 hour before target sleep onset, the rise in melatonin levels in the patient can coincide with the target sleep onset time.

The term ‘24 hours minus the target sleep duration’ may correspond to an equivalent target sleep hunger. For example, a target sleep duration of 8 hours corresponds to a target sleep hunger of 16 hours. The target sleep hunger may be considered as the target time between the actual or target wake time and the target sleep onset time/bedtime. The target sleep hunger may comprise the optimum sleep hunger for achieving sleep onset. Therefore, determining the dosage time may comprise determining the dosage time based on the target wake-up time, the actual wake-up time, the target sleep hunger, t_{SH}, and the dosage offset time:

t_{dose}=later of(t_{AW},t_{TW})+t_{TSH}−t_{DO}

In further examples, the method may include an intermediate step of determining the target sleep onset time based on the target wake-up time or the actual wake-up time.

Determining the target sleep onset time may comprise determining the target sleep onset time as the dosage time, t_{dose}, as the (later of) actual wake-up time, t_{AW}, or target wake-up, t_{TW}, time plus 24 hours minus the target sleep duration:

t_{dose}=later of(t_{AW},t_{TW})+24−t_{TSD}

Or equivalently, determining the target sleep onset time may comprise determining the target sleep onset time as the dosage time, t_{dose}, as the (later of) actual wake-up time, t_{AW}, or target wake-up, t_{TW}, time plus the target hunger:

t_{dose}=later of(t_{AW},t_{TW})+t_{TSH}

Determining the target sleep onset time may comprise determining the target sleep onset time as from 12 to 20 hours, preferably 16 hours after the actual wake-up time or the target wake-up time. The method may comprise determining the dosage time based on the target sleep onset time, for example, the target sleep onset time minus the dosage offset time. By taking melatonin at dosage offset time prior to the target sleep onset, the rise in melatonin levels in the patient can coincide with the target sleep onset time/target sleep hunger. In this way, the method can control the rise in melatonin levels in synchronisation with an optimum sleep hunger.

In some examples, the method may comprise receiving sleep data comprising an insomnia severity rating and applying a severity offset to the dosage time based on the insomnia severity rating. The method may additionally or alternatively comprise applying the severity offset to the target sleep onset time. For example, a patient with a medium severity rating may exhibit mild insomnia related arousal factors corresponding to a severity offset of 1 hour. A patient with a high severity rating may exhibit severe insomnia related arousal factors corresponding to a severity offset of 2 hours.

In some examples, the method may comprise determining a dosage time based on sleep data other than wake-up data. For example, as discussed above, the method may comprise determining a dosage time based on the target sleep onset time. In some examples, determining a dosage time may be based on the target sleep hunger. Alternatively, or in addition, determining a dosage time may comprise determining an actual sleep hunger based on the sleep data. Determining an actual sleep hunger may be based on an actual sleep onset time and a total sleep duration. The total sleep duration may comprise a night-time sleep duration and a day time sleep duration. Methods according to such examples may still achieve the same effect of anchoring the actual wake-time and dosage time as a patient settles into a regular dosage routine.

The method may further comprise receiving further patient data. The further patient data may comprise one or more of: patient personal data, patient record data, patient physiological data, patient goal data, environmental data, patient drug data and patient activity data.

Receiving the further patient data may comprise receiving the further patient data by one or more of: user input (for example patient entered data), sensor input (for example, data from patient sensors such as a heart rate monitor or data from a system sensor such as a temperature sensor) or network input (for example, patient record data or environmental/weather data may be communicated over a network connection).

Personal data may comprise basic patient information relating to age, gender, weight, BMI etc.

Patient record data may comprise medical records including information related to medical history, co-morbidities, prescription data etc. The patient record data may comprise the patient personal data.

Patient physiological data may comprise data relating to one or more physiological measurements. Such measurements may be taken by a patient, a healthcare provider or by a device, such as an electronic device, such as a smartphone or other handheld device. Example physiological measurements include heart rate, blood pressure, oxygen saturation, respiration rate etc.

Patient goal data may comprise desired sleep data as described above in relation to the sleep data. Patient goal data may further comprise a desired medication goal which may be to stop medication with melatonin.

Environmental data may relate to the patient's environment, such as the patient's local environment. The environmental data may comprise one or more environmental measurements. Suitable environmental measurements may include temperature, humidity, and/or light intensity, such as daily light exposure, electronic light exposure, daily average temperature, maximum/minimum daily temperature, and daily rainfall.

Patient drug data may comprise medication information related to the patient's melatonin prescription, including a dosage amount, frequency and duration. The drug data may comprise a record of actual dose data. The actual dose data may comprise dose times, dose amounts, missed doses etc for the patient. The actual dose data may be entered by patient input or may be received via communication with smart drug packaging. The drug data may comprise further prescription data relating to the patient for other drugs.

The patient activity data may comprise any activity likely to affect a patient's ability to sleep. Example patient activity data includes exercise data, ingestion data including food ingestion data, caffeine ingestion data and alcohol ingestion data, electronic device usage data, sexual activity and relaxation activity data.

The method may comprise determining the dosage time based on the further patient data. As specific non-limiting examples, determining the dosage time may be based on any of:
Ingestion data, including food, alcohol and/or caffeine ingestion data;
Exercise data and associated physiological data, the exercise data including time of exercise, duration of exercise and/or intensity of exercise;
Electronic device usage data including a time of usage and/or a duration of usage;
Light exposure data including time and duration of natural light exposure and electronic light exposure; and
Actual dosage data, such as missed doses and dose timings including doses taken early or late.

Ingestion of food late in a day can delay or slow a rise in melatonin levels following exogenous administration. Therefore, determining the dosage time may comprise adjusting the dosage offset time.

In examples, where the method comprises receiving sleep data comprising a target wake-up time, the method may comprise setting the target wake-up time. The target wake-up time is preferably between 5:30 am and 8:30 am. This is because it ideally aligns with the external zeitgebers that help synchronise the circadian rhythm in particular the natural daylight occurrence and light intensity. The method may comprise setting the target wake-up time based on the sleep data and/or the further patient data. In some examples, the method may comprise setting the target wake-up time based on one or more of: a desired wake-up time, a desired sleep duration, and a desired sleep onset time of the patient goal data. In some examples, the method may comprise setting the target wake-up time based on the sleep data and the patient goal data. For example, a desired wake-up time may be 7 am, but an average actual wake-up time is 4 am. The method may comprise setting the target wake-up time as an intervening value between the desired wake-up time and the actual wake-up time, for example 5:30 am. As supporting the treatment progresses, and the patient's actual wake-up time progresses towards the target wake-up time, the method may comprise updating the target wake-up time based on the actual wake-up time and the target wake-up time.

More generally, the method may comprise updating the target wake-up time based on the sleep data. Updating the target wake-up time may be based on the sleep data, the patient goal data and/or the target wake-up time. Updating the target wake-up time may be based on one or more of: an actual wake-up time, an actual sleep duration and an actual sleep onset time.

In one or more examples, the method may comprise indicating the target wake-up time to the patient and/or clinician.

In some examples, the target wake-up time may be determined externally to a system implementing the method. For example, the patient and/or the clinician may determine the target wake-up time and input the target wake-up time to the system.

In one or more examples, the method may comprise updating a dosage amount of the melatonin as a patient progresses and begins to anchor their wake-up time and develop a more regular sleep pattern synchronised with a circadian rhythm. The method may comprise updating a dosage amount of the melatonin based on the sleep data and/or the further patient data. Updating the dosage amount may be based on a difference between the actual wake-up time and the target wake-up time. The method may comprise increasing the dosage amount if the difference is greater than a first threshold difference, for example 2 hours, for a threshold number of days, for example 14 days or 30 days. Updating the dosage amount may also be based on the actual wake-up time, the target wake-up time and the desired wake-up time. For example, the method may comprise decreasing a dosage amount if a difference between the actual wake-up time and the desired wake-up time is less than a second threshold difference for a threshold number of days or if a difference between the actual wake-up time and the target wake-up time is less than the second threshold difference for the threshold number of days.

The method may further comprise providing one or more therapeutic behavioural recommendations. Providing the one or more therapeutic behavioural recommendations may be to the patient and/or the clinician. Providing the one or more therapeutic behavioural recommendations may be based on the sleep data and/or the further patient data. The one or more therapeutic behavioural recommendations comprise any of: an exercise recommendation (for example a timing, type, duration and intensity of exercise); an ingestion recommendation (for example a timing of meals, an ingestion deadline, a caffeine cap and deadline); a relaxation recommendation (for example, timing of baths, a recommended relaxation technique such as meditation); a day light exposure recommendation (for example, an intensity, wavelength and duration of daylight exposure); and an activity recommendation (for example, an electronic device usage deadline).

Determining the dosage time may be based on a patient compliance with the one or more therapeutic behavioural recommendations.

The term “administered” is one of the art and means that a therapy is provided, or given, to the patient. In relation to the present invention, it may be immaterial how the therapies are administered to the patient. For instance, a therapy may be administered to a patient by a healthcare provider or another third-party. The therapy may be administered by an electronic device, such as a smartphone or other handheld device, either automatically or in direct response to user input from the patient, a healthcare provider or another third-party. Alternatively, the patient may administer the therapy himself or herself, such as by taking tablets or meditating. The electronic device may act on instructions provided by a second electronic device that is located remotely from the electronic device, such as a Cloud-based server, where such instructions are transmitted to the electronic device over a network, e.g. the internet or a cellular network.

When a therapy is a pharmacological therapy, such as the administration of the melatonin, any suitable route may be used to administer said therapy. Preferably the route of administration is by oral, rectal, nasal, topical (including buccal and sublingual), transdermal, intrathecal, transmucosal or parenteral (including subcutaneous, intramuscular, intravenous and intradermal) administration. Preferably the route of administration is by oral administration.

Pharmaceutical compositions comprising melatonin useful in a pharmacological therapy may be formulated in unit dosage form, for instance in tablets and sustained release capsules, and in liposomes. Alternatively, pharmaceutical compositions may be provided as un-dosed gels, liquids and syrups to be dosed (by the patient, third-party or an automatic dosage device) prior to administration. Dosage forms useful in relation to the present invention may be prepared by any methods well known in the art of pharmacy.

As treatment progresses, the methods and melatonin for use according to the various aspects of the invention described above, may comprise the steps of determining the melatonin dosage regimen to additionally comprise indicating the dosage amount.

Suitably the pharmaceutical compositions and dosage forms comprise of from 0.05 to 10 mg, preferably 0.1 to 5 mg, more preferably 1 to 3 mg, e.g. 2 mg of melatonin. Other suitable doses are 0.1, 0.15, and then increasing in increments of 0.25 mg steps to 1 mg. In specific examples, the pharmaceutical compositions and dosage forms comprise for example, 0.2, 0.5, 0.7, 0.9, 1.0, 1.2, 1.4, 1.5, 1.7, 1.9 or 2.0 mg of melatonin.

As treatment progresses, the methods and melatonin for use according to the various aspects of the invention described above, may be in the form of an iterative process in which the initial therapy is administered to a patient, and then additional data related to the patient is processed to provide a modified therapy. This helps to maintain the optimal treatment of the insomnia in the dynamic patient environment.

In one specific aspect, the dosage amount of melatonin may be increased. This may occur, for example, if the treatment is not having the expected or desired effect on the patient i.e. the patient is still not able to get to sleep or maintain sleep in accordance with the therapy plan as described above. Alternatively, in another aspect, the dosage amount of melatonin may be decreased. This may occur, for example, if the treatment is having the expected or desired effect on the patient i.e. the patient is able to get to sleep or maintain sleep in accordance with the therapy plan and achieve the targeted wake up times and total sleep time as described above. As the patient achieves this regular sleep pattern, they may be weaned off the melatonin. For example, the daily dosage amount of melatonin may be reduced each week (for example, by the dose increments described above) until the daily dosage amount is 0 mg, i.e. the patient no longer needs to take melatonin to treat insomnia.

The methods and melatonin for use according to the various aspects of the invention described above may have any of the preferred features described previously in the description for other aspects of the invention.

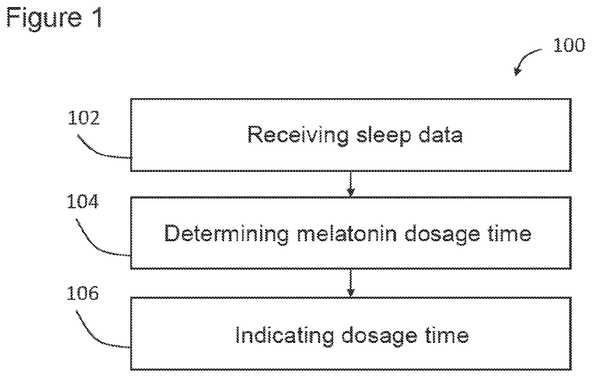

FIG. 1 illustrates a computer implemented method 100 for supporting the treatment of insomnia with melatonin according to the first aspect. The method commences at step 100 by receiving sleep data relating to a patient. Step 104 comprises determining a dosage time for the patient to take a dose of the melatonin based on the sleep data. Step 106 comprises indicating the dosage time. The dosage time may be indicated to the patient and/or the clinician.

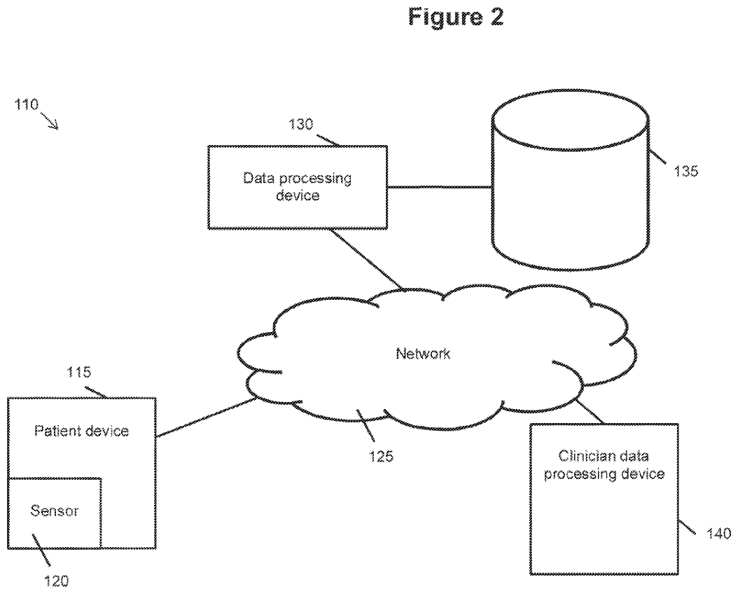

A system 110 suitable for carrying out any of the computer implemented methods disclosed herein methods is shown in FIG. 2. System 110 includes a patient device 115. The patient device 115 may comprise one or more processors configured to: receive sleep data relating to a patient suffering from insomnia; determine a dosage time for the patient to take a dose of melatonin based on the sleep data; and indicate the dosage time.

In the illustrated embodiment, patient device 115 is a smartphone, optionally comprising a sensor 120. However, the invention is not limited in this respect and the patient device 115 can take many other forms, including but not limited to a mobile telephone, a tablet computer, a desktop computer, a voice-activated computing system, a laptop, a gaming system, a vehicular computing system, a wearable device, a smart watch, a smart television, an internet of things device and a medicament-dispensing device.

The patient device 115 may be configured to gather data, including sleep data, relating to a patient and/or the immediate environment. Patient device 115 may gather sleep data and further patient data using sensor 120, which can be any combination of: a light sensor such as a camera, a temperature sensor, an acoustic sensor such as a microphone, an accelerometer, an air pressure sensor, an airborne particulate sensor, a global positioning sensor, a humidity sensor, an electric field sensor, a magnetic field sensor, a moisture sensor, an air quality sensor and a Geiger counter, an electroencephalogram (EEG), a heart rate monitor, a pulse oximeter, or any other sensor capable of gathering polysomnographic relevant data and/or any other characteristic of the patient and/or the patient's immediate environment. Although, the sensor 120 is illustrated as forming part of the patient device 115, in some embodiments the sensor 120 may be separate to, and remotely communicate with, the patient device 115. In other examples, sensor 120 can be omitted from patient device 115.

Alternatively, or in addition, information about the patient and/or the immediate environment of the patient can be obtained via other mechanisms including manual data entry using a human interface device of patient device 115.

The patient device 115 may comprise a memory (not illustrated) for storing the sleep data and/or the further patient data. Such data may also be stored on a database 135 as networked or cloud-based data storage.

The patient device 115 may have one or more applications (or apps) installed on a storage medium associated with the patient device (not shown). The one or more apps may be configured to perform any of the computer implemented methods disclosed herein. The one or more applications may be configured to control data acquisition via sensor 120 and/or to assist the patient in providing data relating to their current condition and/or their immediate environment. The one or more applications may be downloaded from a network, for example from a website or an online application store.

In this example, the system 110 further comprises a data processing device 130 that is communicatively coupled to the patient device 115 via a network 125. In the illustrated embodiment network 125 is the internet, but the invention is not limited in this respect and network 125 could be any network that enables communication between patient device 115 and data processing device 105, such as a cellular network or a combination of the internet and a cellular network.

The data processing device 130 may supplement the patient device 115 and perform one or more steps of any of the computer implemented methods disclosed herein. For example, in some embodiments, the patient device may receive sleep data and provide the sleep data to the data processing device 130. The data processing device 130 may then determine the dosage time for the patient to take melatonin based on the sleep data. The data processing device 130 may then provide the dosage time to the patient device 115 or a clinician device 140 either or both of which may indicate the dosage time. In this way, the data processing device provides networked, server based or cloud based, processing capability to the system for performing the computer implemented methods.

The data processing device may be coupled to a database 135 that can store the sleep data or the further patient data described earlier in this specification.

In this example, the system 100 includes a clinician data processing device 140 that is communicatively coupled via network 125 to the patient device 115 and the data processing device 130. The clinician data processing device 140 may be broadly similar to patient device 115, offering a similar set of functionality. Specifically, the clinician data processing device 140 enables data, including sleep data, relating to the patient and/or the immediate environment of the patient to be collated. Clinician data processing device 140 is contemplated as being physically located at a clinician's premises during its use, such as a sleep clinic, a doctor's surgery, a pharmacy or any other healthcare institution, e.g. a hospital. Clinician data processing device 140 may include one or more sensors like sensor 120, and/or be configured to control one or more separate sensors like sensor 120, which sensors are capable of gathering information about the patient and/or their local environment.

It is also contemplated that clinician data processing device 140 is typically used by a medically trained person with appropriate data security clearance, such that more advanced functionality may be available than via the patient device 115. For example, the clinician data processing device 140 may be able to access a medical history of the patient, generate a melatonin prescription for the patient, place an order for medication, etc. Access to functionality may be controlled by a security policy implemented by a local processor or data processing device 105.

The data processing device 130 and/or the clinician device 140 may an application installed that is compatible with or the same as the application installed on the patient device 115.

It will be appreciated that the various steps of the computer implemented methods disclosed herein may be performed in any combination by any of the one or more processors in the patient device 115, the data processing device 130 and the clinician device 140. For example, all steps may be performed by the clinician device 140 which receives the sleep data from the patient device 115 via the network 125 and optionally the data processing device 130. In a further example, all steps may be performed in a networked back-end on data processing device 130, with patient device 115 and clinician device 140 acting as human interfaces for gathering and indicating data. In a yet further example, all steps may be performed on patient device 115 with clinician device 140 merely gathering relevant data from the patient device 115 for informing or directing the clinician.

It will also be appreciated that one or more of the components of the system 100 could be omitted depending upon the application. For example, at a sleep clinic setting, the disclosed computer implemented methods could be performed solely on the clinician device 140. Alternatively, the methods could be performed solely on the patient device in a domestic setting.

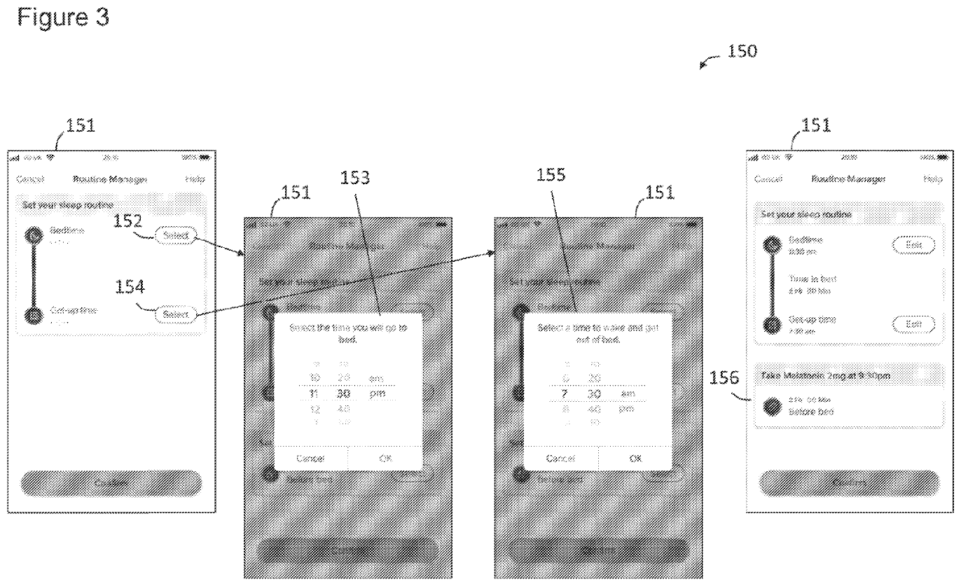

FIG. 3 illustrates a user interface sequence 150 suitable for display on the patient device 115 when the system 110 is carrying out any of the computer implemented methods disclosed herein.

In this example, a display 151 of the user interface sequence 150 comprises select buttons 152, 154 for initiating user input. The select buttons include a target wake time select button 152 and a target sleep onset select button 154.

The target wake time select button 152 responds to user activation by launching a target wake time input 153 of the user interface sequence 150. The target wake time input 153 enables the user to enter the target wake time. In this way, the user interface sequence 150 enables the system to receive sleep data in the form of the target wake time.

The target sleep onset select button 154 responds to user activation by launching a target sleep onset time input 155 of the user interface sequence 150. The target sleep onset time input 155 enables the user to enter the target sleep onset time. In this way, the user interface sequence 150 enables the system to receive sleep data in the form of the target sleep onset time.

Following receipt of the target wake time and/or the target sleep onset time, the system may determine a dosage time for the patient to take the melatonin based on the inputs received at the target wake time input 153 and/or the target sleep onset time input 154. In this example, the system indicates the dosage time to the patient via a dosage time indicator 156 of the display 151.

It will be appreciated that the user interface sequence of FIG. 3 is merely exemplary. As outlined extensively above, the dosage time may be based on other sleep data, such as actual wake time, sleep hunger etc which may be received as, and/or derived from, patient and/or sensor input. The user interface sequence 150 may vary to accommodate all such variations.

The instructions and/or flowchart steps in the above figures can be executed in any order, unless a specific order is explicitly stated. Also, those skilled in the art will recognize that while one example set of instructions/method has been discussed, the material in this specification can be combined in a variety of ways to yield other examples as well, and are to be understood within a context provided by this detailed description.

In some example embodiments the set of instructions/method steps described above are implemented as functional and software instructions embodied as a set of executable instructions which are effected on a computer or machine which is programmed with and controlled by said executable instructions. Such instructions are loaded for execution on a processor (such as one or more CPUs). The term processor includes microprocessors, microcontrollers, processor modules or subsystems (including one or more microprocessors or microcontrollers), or other control or computing devices. A processor can refer to a single component or to plural components.

In other examples, the set of instructions/methods illustrated herein and data and instructions associated therewith are stored in respective storage devices, which are implemented as one or more non-transient machine or computer-readable or computer-usable storage media or mediums. Such computer-readable or computer usable storage medium or media is (are) considered to be part of an article (or article of manufacture). An article or article of manufacture can refer to any manufactured single component or multiple components. The non-transient machine or computer usable media or mediums as defined herein excludes signals, but such media or mediums may be capable of receiving and processing information from signals and/or other transient mediums.

Example embodiments of the material discussed in this specification can be implemented in whole or in part through network, computer, or data based devices and/or services. These may include cloud, internet, intranet, mobile, desktop, processor, look-up table, microcontroller, consumer equipment, infrastructure, or other enabling devices and services. As may be used herein and in the claims, the following non-exclusive definitions are provided.

In one example, one or more instructions or steps discussed herein are automated. The terms automated or automatically (and like variations thereof) mean controlled operation of an apparatus, system, and/or process using computers and/or mechanical/electrical devices without the necessity of human intervention, observation, effort and/or decision.

It will be appreciated that any components said to be coupled may be coupled or connected either directly or indirectly. In the case of indirect coupling, additional components may be located between the two components that are said to be coupled.

In this specification, example embodiments have been presented in terms of a selected set of details. However, a person of ordinary skill in the art would understand that many other example embodiments may be practiced which include a different selected set of these details. It is intended that the following claims cover all possible example embodiments.

### Examples of the Invention

The invention will be further demonstrated by the exemplary case studies below.

### Case Study 1

A 64 year old woman, who worked as a bookkeeper, developed worsening insomnia which she described as “drastically affecting her quality of life”. The woman had reasonably good sleep hygiene and the only relevant medication was an evening dose of 20 mg of amitriptyline taken for pain relief from a rheumatological condition. There is no evidence that amitriptyline affects sleep and it has no interaction with melatonin.

The patient was found to have been experiencing increased stress levels and thus general anxiety. She reported that she has always had some difficulty with her sleep but this had been worse in the previous 12 months following a change in work patterns. The main problem is difficulty getting off to sleep with then some sleep interruptions and difficulty getting back to sleep, with an overall estimate of sleeping only 4 to 5 hours a night.

Specifically the patient described going to bed around 10:30 pm [because it was that time rather than because she was sleepy] and then invariably taking 1 to 2 hours to get to sleep, she did not getting out of bed during that time. Once asleep she then awoke for unclear reasons two or three times and was awake for perhaps 15 minutes each time.

The patient got up usually without the need for an alarm at 6:30 am feeling unrefreshed. In the day she was not excessively sleepy with a score of 1/24 on the Epworth sleepiness scale and scored normally on the General Anxiety scale at 4/7 (both of which are well-known scales in the art). She denied snoring or restless legs.

Treatment: To optimize sleep onset during the initial phase of sleep restriction, which usually takes some days to become effective, she was prescribed 2 mg melatonin to be taken each evening 30-60 mins before bedtime. She was explicitly instructed to take the melatonin at 1100-1130 pm, this time being calculated using the computer implemented method of the invention based on the bedtime recommended as part of a behavioral optimization. Alongside the melatonin treatment, she was introduced to behavioral therapy with the advice to avoid caffeinated drinks after 2 μm and to introduce more exercise to increase body temperature as a drive to sleep. She was encouraged to consider a warm bath one hour before bedtime and to avoid excessive light exposure within the 2 hours before bedtime. In the bedroom time cues were removed and a regular wait time reinforced. Sleep restriction was then introduced advising bedtime first around midnight before later advancing that time incrementally.

As the recommended bedtime gradually was shifted back to a long term optimal time for her of 11 pm, the timing of melatonin administration was shifted in parallel to 1000-1030 pm.

Outcome: Sleep onset within 15 mins of going to bed was achieved with sleep restriction and melatonin. Additionally sleep maintenance and sleep quality was substantially improved. Over a few weeks, bedtime was incrementally advanced with ongoing melatonin taken at the optimized directed time.

### Case Study 2

A 54 year old solicitor, working part time, had increasingly intrusive problems with insomnia. Her sleep had been poor for many years but had significantly worsened since the death of her father six years ago.

She reported going to bed at 10:30 pm after a warm bath at 8 pm, with little difficulty getting off to sleep but then waking 2-5 times a night with significant difficulty getting back to sleep, this being the main problem. Unusually, there was no report of clock watching at these times. She described only sleeping 4-5 hours a night and waking unrefreshed with poor concentration during the day.

The patient had excellent habits regarding sleep hygiene with the avoidance of stimulants and screen time in the evenings. She was physically fit partaking in regular exercise, and had tried hypnosis and acupuncture without benefit.

Her body mass index was reported to be 22.2. There was some anxiety mentioned, but that this was not considered a factor in her sleep problem. Hot flashes were an intermittent problem, but because of a meningioma she had been advised against Hormone replacement therapy (HRT).

Treatment: The patient was prescribed to take melatonin at 930-10 pm. This time was calculated using the computer implemented method of the invention, based on the preferred time to optimally synchronise with the cognitive behavioural therapy.

Outcome: With sleep restriction along with melatonin, the patient's sleep was consolidated. As the treatment went on and her sleep improved, she was weaned off the melatonin. Her overall sleep has improved with her now sleeping poorly only once a week and with ‘life much improved’.

Case study 3

A 38-year-old mechanic with a long, though not lifelong, history of difficulty getting to sleep. This began at the end of school years, in his late teens, before which he had been able to sleep without difficulty between 10 μm and 6:30 am when he would wake easily to an alarm. During his late teens however his sleep delayed until the pattern of sleep described below was established.

Typically, the patient went to bed around 11 pm, because it was that time rather than because he was sleepy, and laid awake with some rumination until vaguely falling asleep between 3 and 4 am. Coffee is needed then to allow the day to begin. Actigraphy confirmed the patient was probably sleeping between the hours of 3 am and 9 am during the week, with a typical weekend extension to noon [or 8 to 9 hours total sleep time, which would be normal].

This man has a chronic insomnia most characteristic of a delayed sleep phase syndrome, which is acquired given the prior normal sleep.

Treatment: Cognitive behavioral therapy I with timed melatonin. The timing of 2 mg melatonin was instructed by the computer implemented method to be 12 h before the body temperature nadir, that is 2 h before the patient's habitual wake time of 12 noon therefore computed as 10 pm, promoting an advance of the ‘clock’ to allow [or guarantee] sleep by 2 am. He was advised to subsequently advance this melatonin timing by 30 mins/week, gradually shifting as his sleep further improved, to an eventual sleep time of 1000-1030 pm.

Patients for case studies 4-6 were seen in a Sleep Disorders Centre. Each patient reported sleep disturbance as the reason for their evaluation.

Case Study 4—Delayed Sleep Onset with Sleep Maintenance Insomnia

A 40 year old schoolteacher with regular poor sleep over the previous two years following a stressful period at work. She described a regular bedtime at 10 pm because of routine rather than feeling sleepy, a characteristic of insomnia of type A. The patient reported a main problem of difficulty getting to sleep until around midnight followed by several waking episodes with some difficulty getting back to sleep. Bedtime had become an issue because of ‘concerns about not getting to sleep or sleeping badly’ and there was a tendency to clock watch. The patient had a need to get up (to an alarm) at a regular time of 7 am in the morning for work, resulting in an estimated sleep time of <6 hrs representing a sleep efficiency of 66%. An Insomnia Severity Index was abnormal at 17. The patient reported impaired concentration at work. There was no history of snoring or restless legs. No daytime naps were taken during the day that would suggest extreme daytime sleepiness. There had been no use of traditional sleep aids. Diagnostic testing included Actigraphy and an accompanying Sleep Diary leading to the conclusion that it was a Chronic Insomnia Disorder.

Treatment: Previous treatment by her primary care physician, including sleep hygiene advice, had been insufficient to affect the desired outcome. After consultation with a sleep physician three strategies were recommended:
1) Cognitive behavioural therapy delivered in part remotely beginning with education about sleep and a recommendation to exercise, as well as cognitive therapy addressing thoughts and feelings.
2) Personalised anchor points of continued regular wake time at 7 am (for work) and retiring to bed at 11:00 pm after a wind down routine.
3) The introduction of melatonin with a dosage time instructed by the computer implemented method to be taken at 8 pm (13 hrs after actual wake time). The rationale in introducing melatonin was that initial behavioural interventions had failed and there was a potential for melatonin to help sleep induction (because of a soporific effect) and to have a direct influence on the earlier timing of the body clock

After 6 weeks of therapy the patient's sleep efficiency improved to >85% and her Insomnia Severity Index was reduced to 7 consistent with her report of consolidated sleep, which persisted after the melatonin was discontinued 6 weeks later.

Case Study 5—Early Waking with Sleep Maintenance Insomnia

A 66 year old patient had a long-standing insomnia (for over 10 years) during which time she had been habituated to zopiclone, which had recently been discontinued following current guidelines, leaving her with very disrupted sleep even in spite of ‘sleep advice’.

Typically, having gone to bed around 10.30 pm, because she is tired or sleepy and not just because of the time, she may get to sleep within half an hour at 11 pm, and then wake briefly between 2 and 3 am if she has been to sleep. She then wakes up in the morning finally at 5 am, remaining in bed until 8 am with a sleep efficiency of <60%.

There was no history of snoring or of restless legs to implicate a physical disturbance and assessment of mood through validated questionnaires (GAD7 for anxiety and PHQ8 for depression) were unremarkable.

Treatment: The current management of these problems through behavioural changes was discussed:
1) Cognitive behavioural therapy delivered in part remotely beginning with education about sleep and the recommendation to exercise at an appropriate time in the day, as well as cognitive therapy addressing thoughts and feelings.
2) Personalised anchor points of a regular earlier wake time of 7 am but retiring to bed as usual at 10:30 pm after a wind down routine.
3) The introduction of timed melatonin with a dosage time instructed by the computer implemented method to be taken at 10:00 pm (30 mins before optimum sleep onset time and 15 hrs after wake time). The rationale in introducing melatonin was that initial behavioural interventions had failed and the potential for melatonin to optimally synchronise with CBT.

After some 4 weeks sleep had consolidated and the wake time extended with a sleep efficiency increase to >85%. After a further 4 weeks melatonin was withdrawn.

### Case Study 6—Sleep Maintenance Insomnia

A patient presented with a long history over many years of difficulty remaining asleep, beginning after a period of post-partum depression. Typically, having gone to bed around 10 pm because of a feeling of sleepiness, she gets to sleep within 15 mins but will then regularly wake between 2 and 3 am for unclear reasons with difficulty getting back to sleep until 5 am, then returning to sleep until woken to an alarm around 7 am. The estimated sleep time was 6 hrs representing a sleep efficiency of 66%. The patient intermittently used antihistamines to help her sleep. The patient used no other sleep aids and had not been subject to behavioural treatment. The patient had some caffeine intake in the form of chocolate. The patient usually exercised in the morning. A sleep study confirmed a sleep efficiency of 60% due to wakening after sleep onset consistent with her complaints but did not reveal any intrinsic pathology that might otherwise explain her insomnia.

### Treatment:

1) Cognitive behavioural therapy delivered in part remotely beginning with education about sleep and a recommendation to exercise at an appropriate time in the day, as well as cognitive therapy addressing thoughts and feelings.
2) Personalised anchor points of a continued regular wake time of 7 am, retiring to bed at 11 pm after a wind down routine.
3) The introduction of timed melatonin with a dosage time instructed by the computer implemented method to be taken at 10 pm (15 hrs after wake time). The rationale in introducing melatonin was that initial behavioural interventions had failed and the potential to consolidate sleep.

After 6 weeks of remote CBT and melatonin the patient's sleep efficiency increased to >90%, approaching her normal sleep requirement of 8 hrs. Melatonin was later withdrawn and at a four month follow up sleep remained sufficient and consolidated.

The above case studies illustrate that controlling a dosage time of Melatonin can provide significant sleep improvements for insomnia patients. In particular, determining a dosage time based on sleep data (e.g. a target wake time, a target sleep onset time etc) can result in improved sleep quality, sleep efficiency and maintenance.

### CONCLUSION

The present inventors have found that tailoring the timing of melatonin administration to the patient's specific issues and sleep targets allowed an effective therapy to be developed to successfully treat their insomnia.

1. A computer implemented method for supporting the treatment of insomnia with melatonin, the method comprising:
receiving sleep data relating to a patient;
determining a dosage time for the patient to take the melatonin based on the sleep data; and
indicating the dosage time.

2. The method of claim 1, wherein the sleep data comprises wake-up time data preferably the wake-up time data comprises one or more of:
an actual wake-up time; and
a target wake up time.

3. (canceled)

4. The method of claim 2, wherein the determining the dosage time comprises determining the dosage time based on:
the actual wake-up time if the actual wake-up time is later than the target wake-up time; and
the target wake-up time if the actual wake-up time is earlier than or equal to the target wake-up time.

5. The method of claim 1 further comprising:
determining a target sleep hunger based on the sleep data; and
determining the dosage time based on the target sleep hunger.

6. The method of claim 5, wherein determining the target sleep hunger comprises determining the target sleep hunger based on a target sleep duration.

7. The method of claim 2, wherein a type of the insomnia is early morning wakening insomnia or sleep maintenance insomnia and determining the dosage time comprises determining the dosage time as from 11 to 19 hours after the actual wake-up time or the target wake-up time or delayed sleep phase insomnia and determining the dosage time comprises determining the dosage time as from 8 to 12 hours after the actual wake-up time or the target wake-up time.

8. (canceled)

9. The method of claim 1, wherein the sleep data comprises one or more of: wake-up time data, sleep duration, sleep onset time, sleep quality, number of waking episodes, duration of waking episodes and day-time sleep data.

10. The method of claim 1 further comprising:
determining an actual sleep hunger based on the sleep data; and
determining the dosage time based on the actual sleep hunger.

11. The method of claim 10, wherein determining the actual sleep hunger is based on a sleep onset time and a total sleep duration, and optionally wherein the total sleep duration comprises a night-time sleep duration and a day-time sleep duration.

12. (canceled)

13. The method of claim 1, wherein the sleep data comprises an insomnia severity rating and the method comprises applying a severity offset to the dosage time based on the insomnia severity rating.

14. The method of claim 1, further comprising:
determining a target sleep onset time based on the sleep-data, preferably based on wake-up time data; and
indicating the target sleep onset time.

15. The method of claim 14, wherein determining the dosage time comprises determining the dosage time based on the target sleep onset time.

16. The method of claim 1, comprising receiving further patient data comprising one or more of: patient record data, patient personal data, patient physiological data, patient goal data, environmental data, patient drug data and patient activity data including ingestion data and optionally determining the dosage time comprises determining the dosage time based on the further patient data.

17-19. (canceled)

20. The method of claim 1, wherein the sleep data comprises a target wake-up time and the method comprises setting or updating the target wake-up time based on the further patient data or on the other data of the sleep data.

21. The method of claim 20, wherein setting the target wake-up time comprises setting the target wake-up time based on patient goal data and/or the sleep data, preferably based on one or more of:
a desired wake-up time of the patient goal data;
a desired sleep duration of the patient goal data; and
a desired sleep onset time of the patient goal data.

22. (canceled)

23. The method of claim 21, wherein updating the target wake-up time comprises updating the target wake-up time based on one or more of:
an actual wake-up time of the sleep data;
an actual sleep duration of the sleep data; and
an actual sleep onset time of the sleep data.

24. The method of claim 23, wherein determining the dosage time comprises determining the dosage time based on the updated target wake-up time.

25. The method of claim 11, comprising updating a dosage amount of the melatonin based on the further patient data or on the sleep data preferably based on a difference between a target wake-up time and an actual wake-up time of the sleep data.

26. (canceled)

27. The method of claim 25, further comprising increasing the dosage amount if the difference is greater than a first difference threshold for a threshold number of days or decreasing the dosage amount if the difference is less than a second difference threshold for a threshold number of days.

28-29. (canceled)

30. The method of claim 1 comprising providing one or more therapeutic behavioural recommendations optionally based on the sleep data and preferably comprising any of:
an exercise recommendation;
an ingestion recommendation;
a relaxation recommendation;
a day light exposure recommendation; and
an activity recommendation.

31-76. (canceled)
